(19) United States
(12) Patent Application Publication
(10) Pub. No.: US 2010/0121172 A1
(43) Pub. Date: May 13, 2010

(54) MICROSCOPIC AND MACROSCOPIC DATA FUSION FOR BIOMEDICAL IMAGING

(75) Inventors: Lance Anthony Ladic, Monmouth Junction, NJ (US); Gianluca Paladini, Skillman, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 12/369,847

(22) Filed: Feb. 12, 2009

Related U.S. Application Data
(60) Provisional application No. 61/113,772, filed on Nov. 12, 2008.

(51) Int. Cl. A61B 5/05

(52) U.S. Cl. 600/407

(57) ABSTRACT

Macroscopic imaging data, such as CT, MR, PET, or SPECT, is obtained. Microscopic imaging data of at least a portion of the same tissue is obtained. The microscopic imaging data is spatially aligned with the macroscopic imaging data. The spatial alignment allows calculation and/or imaging using both types of data as a multi-resolution data set. A given image may include information about the relative position of the microscopically imaged tissue to the macroscopically imaged body portion. This positional relationship may allow viewing of affects or changes at cellular levels as well as less detailed tissue structure or organism levels and may allow determination of any correlation between changes in both levels.

### RELATED APPLICATIONS

The present patent document claims the benefit of the filing date under 35 U.S.C. §119(e) of Provisional U.S. patent application Ser. No. 61/113,772, filed Nov. 12, 2008, which is hereby incorporated by reference.

### BACKGROUND

The present embodiments relate to biomedical imaging, such as medical diagnostic, pharmaceutical, or clinical imaging. Different types of medical imaging modes are available. For example, medical imaging includes x-ray, ultrasound, computed tomography (CT), magnetic resonance (MR), positron emission (PET), single photon emission (SPECT), and optical imaging. Other medical imaging includes microscopy. A tissue sample is scanned, such as taking an optical picture, using magnification available with a microscope.

The biomedical image data may be used to assist medical professionals, such as researchers. For example, a pre-clinical animal or clinical patient trial is performed. Drug discovery and development is a complex, multistage process that is both time consuming and expensive. A large percentage of overall drug R&D costs are attributed to attrition, the failure of drug candidates to progress through the pipeline. The vast majority of these failures occur in the discovery and preclinical phases of drug discovery, which comprise basic research, target identification and validation, and screening and optimization of drug candidates.

Before drug candidates can progress to human clinical trials, the drugs are typically validated in cellular and animal models. The correlation between how a candidate drug behaves within cells (at the most basic level) and within a model organism (such as a lab animal) is important for understanding the drug's effects and/or mechanism of action in relationship to structural and functional components within living systems.

The relationships between cellular and organism-level function is also a component for increasing understanding of systems biology. In addition to progressing basic scientific knowledge, this could lead to novel translational diagnostic and therapeutic approaches.

To assist in analysis, a patient is imaged. For example, tissue is imaged to determine the effect, if any, of a candidate drug on the tissue. For a given mode of imaging (e.g., CT), different renderings may be provided at different resolutions. More than one mode of imaging may be used to assist in analysis. However, the data is obtained and analyzed separately.

### BRIEF SUMMARY

By way of introduction, the preferred embodiments described below include methods, systems, instructions, and computer readable media for biomedical imaging or other study. Macroscopic imaging data, such as that from a CT, MR, PET, or SPECT scanner, is obtained. Microscopic imaging data of at least a portion of the same tissue is obtained. The microscopic imaging data is spatially aligned with the macroscopic imaging data. The spatial alignment allows calculation and/or imaging using both types of data as a multi-resolution data set. A given image may include information about the relative position of the microscopically imaged tissue to the macroscopically imaged body portion. This positional relationship may allow viewing of affects or changes at cellular levels as well as less detailed tissue structure or organism levels and may allow determination of any correlation between changes in both levels.

In a first aspect, a method is provided for biomedical imaging. Microscopic data representing a first region of tissue is obtained. Macroscopic data representing a second region of tissue is obtained. The second region is larger than the first region. The microscopic data and the macroscopic data are spatially aligned. An image is generated as a function of the microscopic data, macroscopic data, or both microscopic and macroscopic data and as a function of the spatial aligning.

In a second aspect, a system for biomedical imaging is provided. A memory is operable to store first data representing a tissue volume. The first data is from a microscopic imaging source. The memory is operable to store second data representing the tissue volume. The second data is from a macroscopic imaging source of a different type than the microscopic imaging source. The first data has a greater resolution than the second data. A processor is operable to register the first data and the second data and operable to render an image as a function of the first and second data. A display is operable to display the image of the tissue volume.

In a third aspect, a computer readable storage medium has stored therein data representing instructions executable by a programmed processor for biomedical study. The storage medium includes instructions for registering microscopy scan data with macroscopy scan data. The microscopy scan data represents a first tissue region that is a sub-set of a second tissue region represented, with lesser resolution, by the macroscopy scan data. The instructions are also for determining quantities from the registered microscopy and macroscopy scan data at different resolutions and for modeling as a function of the quantities.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for biomedical imaging and/or study; and

FIG. 2 is a flow chart diagram of one embodiment of a method for registering microscopic and macroscopic data in biomedical imaging.

### DETAILED DESCRIPTION OF THE DRAWINGS AND PRESENTLY PREFERRED EMBODIMENTS

Software integrates both microscopic and macroscopic biomedical imaging data for the purpose of visualization analysis. In particular, microscopic and macroscopic biomedical imaging data are acquired from different sources. The data may include multiple overlapping, multispectral (e.g. multi-label fluorescence, in the case of microscopy) or multi-modality (e.g. PET/SPECT/CT data, in the case of macroscopic data) datasets. The microscopic and macroscopic datasets are registered (i.e. aligning a microscopic image/volume within a related macroscopic image/volume). The registered data is used for viewing, manipulating, or navigating. For example, datasets associated with objects, structures, and/or function (e.g., labeled for a targeted protein) within the micro and macro datasets are selected. The dataset may be used for rendering at different resolution scales (“multi-resolution viewing”).

The integration of microscopic and macroscopic biomedical imaging data and the ability to view, manipulate, navigate, and/or analyze this data may permit the correlation of structure and/or function at different resolutions. This correlation may further the understanding of systems biology, such as how molecular or cellular structure and/or function relate to tissue, organ or whole organism structure and/or function. The information derived may aid the understanding of disease or in the development of diagnostic tests or therapeutics (i.e. drugs).

In one embodiment, imaging software handles both macroscopic and microscopic imaging data. The software is bundled with existing hardware (microscopes and/or small animal imaging equipment) or sold as accessory software that could be purchased separately. Biotech or pharmaceutical companies may use the software or workstation for drug and contrast/imaging agent discovery or development. Academic or biomedical research may use the software or hardware for basic life science research (e.g. physiology, anatomy, pharmacology, genetics, etc.). An example application is neurology. The aligned data is used for examination of neurodegenerative diseases such as Alzheimer's and Parkinson's. The aligned data may be used for neuroanatomical tracing studies, correlating neural connectivity within the brain and/or from distal organs/tissues with observed functional activity. Another example application is oncology, such as for imaging of tumors and/or surrounding blood supply. The registration of micro and macro data may be used in connection with small animal imaging, development of radiopharmaceuticals or other imaging agents, diagnosis, or other uses.

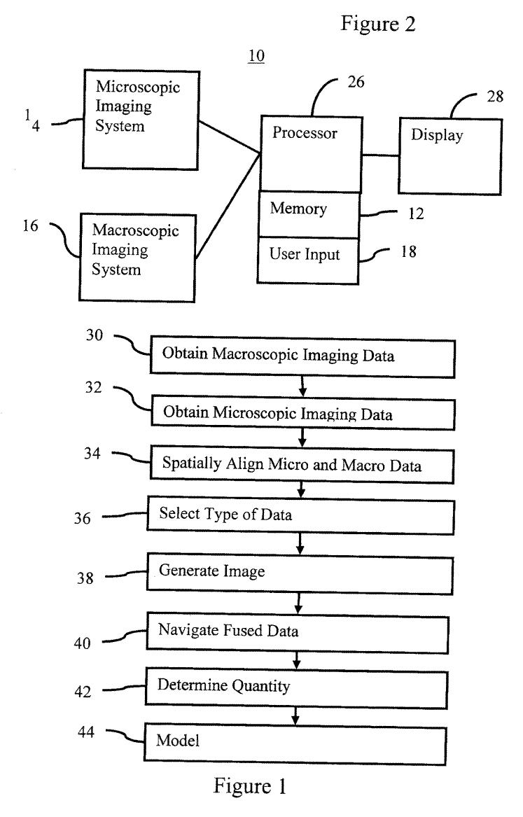

FIG. 1 shows a method for biomedical imaging. The method is implemented by the system 10 of FIG. 2 or another system. The acts are performed in the order shown or other orders. For example, acts 42 and/or 44 are performed before act 38. Additional, different, or fewer acts may be provided. For example, act 36, 40, 42, and/or 44 are not provided. As another example, acts 38 and 40 are not provided.

In act 30, macroscopic data is obtained. Macroscopic data is data representing gross tissue structure or an organism, but not at cellular, sub-cellular, or molecular resolutions or of cellular structure. Expressed relatively, the macroscopic data has less resolution than microscopic data.

Macroscopic data is obtained with a different imaging modality than microscopic data. For example, the macroscopic data is image or scan data acquired using x-rays, ultrasound, magnetic resonance, photon emission, positron emission, or other radio frequency energy. Any now known or later developed type of scanning or mode may be used, such as computed tomography, magnetic resonance, x-ray, ultrasound, positron emission tomography, single photon emission tomography, or combinations thereof.

The macroscopic data is obtained from an imaging system. For example, 2D, 3D, and/or 4D image data is acquired in real-time from radiological equipment, such as CT, MR, micro-MR, PET, micro-PET, SPECT, SPECT-CT, ultrasound, or X-Ray systems. Alternatively, the macroscopic data is acquired from memory, such as from an image storage server or database. Either single or multi-modality (e.g., CT and MR) image data is acquired and stored for further registration with microscopic imaging data.

The macroscopic data represents a region of a patient, such as tissue and/or fluid. The region is a planar region (e.g., 2D) or a volume region (e.g., 3D). For example, macroscopic data spaced along a regular grid in three-dimensions is obtained. Alternatively, the data may be spaced according to a scan format. Due to the lesser resolution, the macroscopic data may represent a larger region than the microscopic data. In other embodiments, the macroscopic and microscopic data represent a same size region.

The macroscopic data is obtained for study of a specific patient, animal, and/or tissue. In one embodiment, the macroscopic data is acquired for study of a candidate drug. The data is pre-clinical data (i.e. animal imaging) or clinical data (human patients). The data represents a scan prior to and/or after exposure to the candidate drug. For example, the macroscopic data is acquired by scanning or imaging before and after exposure to the drug in order to determine the effects the drug may have had on tissue structure or function. As another example, the macroscopic data is obtained from a patient for diagnosis of a medical problem. The tissue is scanned while still within (e.g. internal organs) or on (e.g. skin) the patient. In another example, the tissue is scanned outside of or after being biopsied/removed from a patient.

The data may be segmented to identify particular tissue structures, landmarks, or organs. Automated, semi-automatic, or manual segmentation may be used.

The scan may be performed to better indicate function of the tissue. For example, the data is responsive to imaging agent labeling. An imaging or contrast agent, such as FDG (radiolabeled fluorodeoxyglucose) for PET, is applied prior to scanning. The scanning is performed to sense the imaging agent. For example, FDG may be used in conjunction with PET scanning to investigate the functional pattern or distribution of glucose metabolism in the tissue. Other examples include imaging agents designed to bind to specific proteins or other molecules, and data responsive to a scan to detect such imaging agents. In other examples, a dye or chemical is injected, ingested or topically applied to allow detection for a scan. Any now known or later developed labeling for function may be used.

In one embodiment, fiduciary markers are provided by or in the scanned tissue or patient. The markers are positioned prior to acquisition of the macroscopic and microscopic data. Any fiduciary marker may be used, such as beads, buttons, or other materials selected to be responsive to the scan for macroscopic data. Alternatively, a lack of material may be used. For example, a fine needle creates holes through the region of interest.

The fiduciary markers are located to indicate position. For example, a line and a point, or three points are positioned for accurate orientation and registration of the region of interest. The markers are within the tissue, adjacent the tissue, or spaced from the tissue. For example, the markers are positioned on the skin of a patient. The macroscopic scan coordinate system is aligned with the markers or includes the markers for later alignment.

In alternative embodiments, features within the tissue itself (e.g. blood vessels or other morphological landmarks) are used as markers. These tissue features assist with the registration instead of or in addition to fiduciary markers.

In act 32, microscopic data is obtained. Microscopic data represents micron or sub-micron levels of resolution. Microscopic data represents cellular or molecular information (i.e. structural or functional). The microscopic data has a greater resolution than the macroscopic data.

The microscopic data represents a region of tissue. The region is a sub-set of the region for the macroscopic data, but may represent regions outside of the macroscopic scan or the same sized region. The region is a two or three-dimensional region. For example, data representing tissue along a regularly spaced or scan distributed three-dimensional grid is obtained.

Microscopic data is obtained with a microscope or other device for imaging at micron levels of resolution. Any modality may be used, whether now known or later developed. The modality used for acquiring the microscopic data is a different mode than used for acquiring the macroscopic data.

In one example, histology and/or immunocytochemistry is performed on the appropriate region of interest. In the case of pre-clinical data, an animal is euthanized and perfused. For non-live preparations, the animal is typically fixed (e.g., with paraforrnaldehyde) before histological processing. In the case of clinical data, a patient's organ or tissue sample is usually either removed or biopsied, but “in vivo” (in living system) imaging (e.g. using fiber optic imaging methods) could also be used. Removed organs, such as a prostate, are further processed for histology. During histological processing, thick tissue sections (e.g. 50-100 microns) are cut along a desired planes (coronal, saggital and/or longitudinal) through the region of interest. The tissue section is alternatively oriented with respect to fiduciary markers, such as being parallel to a plane established by the markers, being through the markers, including the markers, or at a measured angle or position relative to the markers.

The prepared tissue is scanned or imaged to obtain the microscopic data. For example, confocal microscopy is performed to obtain microscopic data representing the tissue region as a three-dimensional region. The harvested tissue sections are scanned with a microscope. The microscope acquires 2D, 3D, and/or 4D microscopic data sets. In confocal scans, data representing different planes throughout the tissue section are acquired. Other modalities, now known or later developed, may be used, such as a scanning electron microscope.

In one embodiment, one or more sets of the microscopic data are functional data. For example, the tissue is incubated with fluorescently labeled or chromogenically labeled antibodies. The antibodies are used to label the desired targets. For example, multiple fluorophores/chromophores label more than one functional structure of interest (i.e., multispectral imaging). The microscopic data may provide a more detailed representation of structural or functional information that was captured by related macroscopic data. For example, microscopic data may permit (sub-)micron resolution localization and visualization of radiopharmaceuticals or other imaging agents used in a macroscopic imaging procedure that have been taken up by, or are bound to, cells in the target area. The labeling co-localizes the cells with other sub-cellular components of interest (e.g. receptors, neurotransmitters, structural elements, etc.). Data for multiple images and/or volumes is acquired (e.g. one image or volume per fluorophore/chromophore). Alternatively, a single volume that contains the locations of multiple fluorophores/chromophores is obtained. In other embodiments, a single volume of single function data is obtained.

The microscopic data is obtained as “in vitro” or “in vivd” imaging data. The data is obtained from memory or in real time with scanning. The data represents the tissue before and/or after therapy, before and/or after exposure to a candidate drug, or after biopsy for diagnosis.

The microscopic data may represent fiduciary markers. For example, the fiduciary markers reflect the energy used to scan the tissue, such as being optically detectable. By sectioning the tissue to include the markers on or within the tissue, information representing the markers as well as the tissue is obtained. In alternative embodiments, the microscopic data does not represent the markers, such as where morphological features or speckle pattern are used for alignment.

In one embodiment, at least some of the microscopic data is scanned and/or prepared for registration. The data is different from data used for imaging or other purposes. For example, reference tissue sections are cut and exposed to a standard histological stain (e.g. hematoxylin and eosin), and digitized images of these sections are acquired at one or more magnifications (e.g. 100×, 400×, 1000×). The resulting microscopic data is used to provide structural reference for later registration of the microscopic data with the macroscopic data.

In act 34, the microscopic data and the macroscopic data are spatially aligned. The microscopy scan data is registered with the macroscopy scan data. The registration orients the coordinate systems for the different types of data. The microscopy scan data represents a tissue region that is a sub-set of a tissue region represented, with lesser resolution, by the macroscopy scan data. The location of the sub-set is determined. For three-dimensional imaging, the voxel's spatial locations representing the same region are identified.

Registering is performed along two or three-dimensions. Inter-modality 3D-3D registration may provide registration that is more accurate than 2D-3D or 2D-2D. The registration accounts for rotation or translation along any number of the dimensions. Any combination of translation and rotation degrees of freedom may be used, such as 6 degrees (3 axes of rotation and 3 axes of translation).

The data is registered using tissue landmarks (e.g. morphological features), fiduciary markers, sensor measurements, data matching, correlation, atlases, or combinations thereof. For example, tissue landmarks and/or fiduciary markers common to both of the macroscopic and microscopic datasets are aligned. As another example, the location of the microscopically scanned tissue relative to fiduciary markers is aligned relative to the locations of the fiduciary markers represented by the macroscopic data. In another example, a stereotactic atlas or other atlas indicates the relative location of landmarks or other information represented by the microscopic data to an organ or structure represented by the macroscopic data. Various types of atlas data (e.g. for brain, across different species) is available. The spatial position of the microscopic volume is provided in relation to surrounding anatomical and/or functional structures or landmarks. This provides the viewer with a frame of reference for the location of the microscopic volume.

The alignment is performed manually or semi-automatically. For example, the user indicates landmarks or markers common to both datasets. A processor then spatially aligns based on the landmarks or markers. The regions represented by the two data sets are translated, warped, and/or rotated to position the same landmarks or markers in the generally same positions. As another example, the user indicates the rotation and/or translation to align the regions represented by the macro and microscopic data.

Alternatively, automatic image processing determines the alignment. In one embodiment, the data sets are correlated. For example, a data pattern, landmarks, or fiduciary markers in the different datasets are correlated. By searching through different translations, warpings, and/or rotations, the alignment with a highest or sufficient correlation is selected. Any search pattern may be used, such as numerical optimization, course-to-fine searching, subset based searching, or use of decimated data.

The correlation may be based on all of the data in the sets. Alternatively, the correlation is based on a sub-set. The sub-set may be the reference frames of microscopic data or data for at least one feature represented in the both types of data. For example, the user or a processor identifies features in each data set. The features may be tissue boundaries, tissue regions, bone region, fluid region, air region, fiduciary markers, combinations thereof, or other feature. The data representing the features with or without surrounding data is used for the correlation. The features may be identified in one set (e.g., microscopic) for matching with all of the data in another set (e.g., macroscopic), or features of one set may be matched to features of another set.

The data may be used for correlation without alteration. In other embodiments, one or both sets of data are filtered or processed to provide more likely matching. Filters may be applied to highlight or select desired landmarks or patterns before matching. For example, higher resolution microscopic data is low pass filtered, decimated, or image processed to be more similar to macroscopic data. As another example, gradients for each type of data are determined and matched.

The macroscopic data may be sensitive to heart, breathing or other motion. To eliminate or reduce the respiratory motion from the data to be registered, the patient may be asked to hold their breath. Alternatively, the macroscopic data is associated with a phase of the breathing cycle associated with relaxation of the tissue or strain on the tissue most similar to the tissue as scanned for the microscopic data. A similar approach may be used to deal with heart motion.

In one embodiment, the registration process computes a rigid (i.e., translation and/or rotation without warping) transformation from the coordinate systems of the microscopic data and the macroscopic data. In another embodiment, a non-rigid transform is applied. The tissue may be subject to very different forces between the scanning for macro and microscopic data. For example, preparing the tissue for microscopic imaging results in separation from other tissues and compressive forces not applied to the tissue while in the patient or animal. To account for the different forces, non-rigid registration may expand and/or contract the coordinate systems and/or variance of the expansion and contraction along one or more axes. Due to tissue warping during histology and/or immunocytochemistry, non-rigid registration algorithms may better match the histological sections with the macroscopic imaging scans.

The spatial alignment is used to form one set of data. For example, the two data sets are fused. The resolution in the fused data set may vary, such as having higher resolution for the region associated with the microscopic data. Alternatively, the spatial relationship of the macro and microscopic datasets is used, but with separately stored data sets.

One alignment may be used for other combinations of data. For example, both CT and MR macroscopic datasets are obtained. If the coordinate systems are the same or have a known relationship, the alignment of the CT data with the microscopic data may also be used to indicate the alignment for the MR macroscopic data with the microscopic data. The alignment of data acquired with no or one type of labeling (e.g., stain, imaging agent, biomarker, or other functional indicator) may be used to align datasets acquired with other types of labeling.

In act 36, one or more types of macro and/or microscopic data are selected. The selection is performed by the user or by a processor. Where multiples types of micro or macroscopic data are obtained, one or more may be selected. For example, data representing one tissue function is selected. The micro and/or macroscopic data for quantification, analysis, and/or imaging are selected. More than one type of data may be selected, such as for determining quantities or rendering images for different types of data. The function selected for the microscopic data may be different than or the same as selected for the macroscopic data.

In act 38, an image is generated. The image is a two-dimensional representation rendered from data representing a volume. Any type of three-dimensional rendering may be used, such as surface or projection rendering. Any type of blending or combination to data may be used. Alternatively or additionally, a two-dimensional image representing a plane or surface is generated. Data along or near the plane may be interpolated or selected, allowing generation of an image representing any arbitrary plane through a volume. A multi-planar reconstruction may be generated. Images for fixed planes, such as associated with a plane defined by fiduciary markers, may be generated.

The image is generated as a function of the spatial aligning of act 34. The spatial alignment allows indication of the position of the microscopic data relative to the macroscopic data. For example, an overlay or more opaque region in an image generated from macroscopic data indicates the relative location of available microscopic data. The spatial alignment allows generation of the image from both types of data. For example, the macro and microscopic data are interpolated and/or decimated to a same or similar resolution. The image is generated using both types of data. The data may be relatively weighted, such as by assigning an opacity value. The different types of data may be rendered differently and overlaid with each other. The different types of data may be used for different pixel characteristics, such as macroscopic data indicating intensity and microscopic data indicating color or shade. The spatial alignment determines which values represent which voxel or spatial locations.

The image is generated as a function of the microscopic data, macroscopic data, or both microscopic and macroscopic data. The image may be rendered from values selected from one or both types of data. For example, separate images may be rendered for the macro and microscopic data, but with an overlay or indication of the relative positioning.

In one embodiment, the rendering is performed as a function of a zoom level. A low-resolution (e.g., low zoom) image may be rendered from macroscopic data. The location of the microscopically scanned tissue may be included, such as providing an overlay or higher resolution region. This indicates the relative position of the microscopic scan to the macroscopic scan. A high-resolution (e.g., high zoom) image may be rendered from microscopic data. A range of middle resolution images may be rendered from both macro and microscopic data. The rendering may indicate the relative position of the microscopic scan region to the macroscopic scan region. As the user zooms into the region of the microscopic sub-volume, the surrounding macroscopic volume may be rendered more transparently, becoming abstracted. For example, the macroscopic data is rendered as a simple, semi-transparent surface volume showing surrounding anatomical landmarks. The microscopic volume detail progressively increases when zooming in (e.g. using different volume texture resolutions).

In one embodiment, any now known or later developed multi-resolution imaging may be provided. Multi-resolution, multi-scale imaging visualizes the fused data at different zoom levels. At the macroscopic level, the microscopic image or volume data is overlaid or included in the form of a rectangular sub-region at the appropriate position and orientation. As the user zooms into the region of the microscopic sub-region, the surrounding macroscopic image or volume data is visualized together with the surrounding anatomical landmarks. The microscopic image or volume detail is progressively increased when zooming. A variable level of detail rendering may permit visualization between microscopic and macroscopic scales, allowing the user to view relative differences and effects at different scales of a given drug, disease, and/or therapy.

In an alternative embodiment, a wire frame or graphic represents the microscopic region in an image from the macroscopic data. A separate microscopic image is generated for the microscopic region. For three-dimensional rendering, the projection or viewing direction is the same or different for both images. Alternatively, the spatial alignment is used to overlay rendered or generated images.

In act 40, the user navigates using the macroscopic and microscopic data. After an image is generated, the user may indicate a different viewing direction, zoom level, opacity weighting, and/or other rendering parameter. Subsequent images are generated based on the changes. The user may navigate to more closely examine are given region, such as zooming into view a smaller region at greater detail. The image generation may access sub-sets of data as needed based on the navigation to limit processing and/or transfer bandwidth. As the user navigates to different zoom levels and/or sub-regions, the data appropriate for the zoom level and sub-region is used to generate the image. Different zoom levels may correspond to different relative amounts of the microscopy and macroscopy scan data. For example, a low-resolution image may use mostly macroscopic data with microscopic data being used to render a small section. A high-resolution image zoomed to the microscopic scan region may use mostly microscopic data with low opacity macroscopic data indicating surrounding tissue. Other levels of zoom may use equal or different amounts of the macro and microscopy scan data depending on the size and relative position of the imaged region of interest to the microscopic scan region.

In act 42, one or more quantities are determined. Any quantity may be determined. For example, area, volume, number of voxels, average, variance, statistical value, or other value is determined. The data may be filtered to better highlight or emphasize values representing the desired characteristic for quantification. Any now known or later quantification may be used. The same or different quantities are calculated from the macroscopic and microscopic data.

The quantities are determined from the microscopy scan data of the selected type and/or other functional types. Quantities may be determined from macroscopy data. The registration of the macroscopy and microscopy data may be used to determine the region of interest for which the quantities are calculated.

The obtaining of acts 30 and 32 and spatial alignment of act 34 may be repeated. Other acts may be repeated as well. The repetition occurs at different times. For example, macroscopic and microscopic data is obtained and aligned before and after exposure of tissue to a drug. The repetition allows for temporal correlation. The change or progression of disease (e.g., before and after therapy) and/or reaction to drug exposure may be determined at macro and microscopic levels.

The temporal correlation may be indicated by change or difference between the same quantity calculated for different times. For example, a volume or average intensity associated with a labeled function is calculated from data representing tissue prior to exposure to a drug and from data representing tissue after exposure to the drug. A time series of values may be determined to show progression. Correlation analysis between microscopic and macroscopic data may also be provided.

In act 44, the correlation, temporal change, other change, and/or tissue are modeled. Any type of modeling may be used, such as a machine trained or learned model. The quantities are used to model the tissue. The tissue change indicates the tissue response to therapy, disease, and/or drug exposure. The quantities may allow better prediction of the tissue response in other situations. For example, changes are quantified at the microscopic level with microscopic functional imaging data (e.g. the change before and after application of a drug). As another example, the distribution of and quantity of one or more sub cellular components (e.g. receptors) is quantified and provided with functional macroscopic observations.

FIG. 2 shows a system 10 for medical imaging. The system 10 includes a memory 12, a microscopy system 14, a macroscopy system 16, a user input 18, a processor 26, and a display 28. Additional, different, or fewer components may be provided. For example, a network or network connection is provided, such as for networking with a medical imaging network or data archival system. As another example, additional macroscopy and/or microscopy systems are provided. In another example, the microscopy and/or macroscopy systems 14, 16 are not provided. The marcroscopy and/or microscopy data are stored in the memory 12.

The processor 26, user input 18, and display 28 are part of a medical imaging system, such as the diagnostic or therapy ultrasound, fluoroscopy, x-ray, computed tomography, magnetic resonance, positron emission, or other system. Alternatively, the processor 26, user input 18, and display 28 are part of an archival and/or image processing system, such as associated with a medical records database workstation or server. In other embodiments, the processor 26, user input 18, and display 28 are a personal computer, such as desktop or laptop, a workstation, a server, a network, or combinations thereof. The memory 12 is part of the workstation or system or is a remote database or memory medium.

The user input 18 is a keyboard, button, slider, knob, touch screen, touch pad, mouse, trackball, combinations thereof, or other now known or later developed user input device. The user input 18 receives user indication of interaction with a user interface. The user may select data, control rendering, control imaging, navigate, cause calculation, search, or perform other functions associated with use, imaging, and/or modeling of macroscopic and microscopic data.

The memory 12 is a graphics processing memory, a video random access memory, a random access memory, system memory, random access memory, cache memory, hard drive, optical media, magnetic media, flash drive, buffer, database, server memory, combinations thereof, or other now known or later developed memory device for storing data or video information. The memory 12 is part of an imaging system, part of a computer associated with the processor 26, part of a database, part of an archival system, part of another system, or a standalone device.

The memory 12 stores one or more datasets representing a two or three-dimensional tissue volume. The tissue volume is a region of the patient or animal, such as a region within the chest, abdomen, leg, head, arm, or combinations thereof, or a region of biopsied or harvested tissue. The tissue volume is a region scanned by a medical imaging modality. Different modalities or even scans with a same modality may be of a same or different size regions with or without overlap. The data may represent planar (2D), linear (1D), point, or temporal (4D) regions for one or more datasets.

At least one set of data is data from a microscopic imaging source, such as the microscopic system 14. The microscopic system 14 is a microscope, confocal microscope system, or other now known or later developed microscopic imaging system.

At least one set of data is data from a macroscopic imaging source, such as the macroscopic system 16. The macroscopic system 16 is an ultrasound, x-ray, MR, CT, PET, SPECT, or other now known or later developed macroscopic imaging system. The macroscopic system 16 is different than the microscopic system, so that the data are from different modalities and/or imaging sources.

The macroscopic and/or microscopic data represent the tissue prior to, after, and/or during treatment, drug exposure, and/or disease. The microscopic data has a greater resolution than the macroscopic data. Any relative differences in resolution may be provided. Due to the differences in resolution, the macro and microscopic data represent tissue structure at different levels. The macroscopic data represents the tissue at a larger structure level than the microscopic data.

The macroscopic and microscopic data is in any format. For example, each data set is interpolated or converted to an evenly spaced three-dimensional grid or is in a scan format at the appropriate resolution. Different grids may be used for data representing different resolutions. Each datum is associated with a different volume location (voxel) in the tissue volume. Each volume location is the same size and shape within the dataset. Volume locations with different sizes, shapes, or numbers along a dimension may be included in a same dataset. The data coordinate system represents the position of the scanning device relative to the patient.

In one embodiment, one or more microscopic and/or macroscopic datasets include labeled tissue function information. The scan and/or processing of the data are performed to isolate, highlight, or better indicate tissue structure, locations, or regions associated with a particular function. For example in fluoroscopic imaging, an imaging agent (e.g., iodine) may be injected into a patient. The imaging agent provides a detectable response to x-rays. By flowing through the circulatory system, the imaging agent may provide detectable response highlighting the circulatory system, such as the vessels, veins, and/or heart. As another example, multispectral confocal microscopic imaging generates a plurality of data sets each representing different structural or functional aspects associated with the tissue. Molecular level labeling may be used, such as exposing the tissue to fluorescently or chromogenically labeled antibodies designed to bind to particular cellular or tissue structure or proteins. These antibodies are designed to be visible in the scanning method.

The memory 12 or other memory is a computer readable storage medium storing data representing instructions executable by the programmed processor 26 for medical study, such as modeling and/or imaging. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 26 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining position, modeling, and/or generating images. The processor 26 is a single device or multiple devices operating in serial, parallel, or separately. The processor 26 may be a main processor of a computer, such as a laptop or desktop computer, or may be a processor for handling some tasks in a larger system, such as in an imaging system.

The processor 26 loads the data. Depending on the zoom level of the image to be rendered, the processor 26 loads the appropriate data. For example, all or a sub-sampling of the macroscopic data is loaded for little to no zoom levels. Microscopic data may be not be loaded for such zoom levels. For greater levels of zoom, only the sub-set of macroscopic data within a zoomed region is loaded. The microscopic data is loaded for zoom levels for which the microscopic data contributes to the rendering. Sub-samples may be loaded to avoid transfer bandwidth or processing bandwidth burden. Any multi-resolution imaging and associated data loading may be used.

The processor 26 also loads the micro and macroscopic data for registering. Reference data, rather than an entire set of data, may be loaded and used for registering. Alternatively, the entire dataset is used. The spatial alignment in rotation, translation, and/or warping of the macro and microscopic data is determined.

The registration is performed as a function of tissue structure represented in both types of data, fiduciary markers represented in the both types of data, functional pattern represented in both types of data, atlas information, or combinations thereof. For example, similarities between the microscopic data and the macroscopic data are identified. Image processing may identify features. The user may identify features. Identifying three or more features or one or more features with a corresponding orientation represented by both data sets indicates relative positioning of the volumes.

Alternatively, similarity is determined using a correlation, such as a minimum sum of absolute differences, cross correlation, autocorrelation, or other correlation. For example, a two or three-dimensional set of data is translated and/or rotated into various positions relative to another set of data. The relative position with the minimum sum or highest correlation indicates a match, alignment, or registration location. The set of data may be sub-set, such as a region of interest or a decimated set, or may be a full set. The set to be matched may be a sub-set or full set, such as correlating a decimated region of interest sub-set of microscopic data with a full set of macroscopic data.

The relative positioning indicates a translation, warping, and/or rotation of one set of data relative to another set of data. The coordinates of the different volumes may be aligned or transformed such that spatial locations in each set representing a same tissue have a same or determinable location. The registration for one set of microscopic data with macroscopic data may indicate the registration for other sets of the microscopic and/or macroscopic data.

The processor 26 is operable to render an image as a function of the registered data. Any type of rendering may be used, such as surface rendering, multi-planar reconstruction, projection rendering, and/or generation of an image representing a plane. For example, the image is generated as a rendering of or an arbitrary plane through the tissue volume. The image includes values for pixel locations where each of the values is a function of one or both of macro and microscopic data. For example, the macroscopic data is interpolated to a higher resolution and the microscopic data is decimated to a lower resolution such that the two resolutions match. The image is generated from both types of data.

The image is rendered based on user selection of the type of data. Where datasets corresponding to different or no structural or functional labeling are available, the user may select the dataset to be used for imaging. The dataset may be the same or different from the data used for registration.

The image is generated as a function of the zoom level. The user or the processor 26 indicates the zoom level. The data appropriate for that zoom level is selected and used for generating the image using any now known or later developed multi-resolution imaging.

Where both macro and microscopic data are used to generate the image, the types of data are blended. The blending may be a function of the zoom level. For example, greater zoom levels may emphasize the microscopic data, weighting the macroscopic data with a lesser weight.

Spatially aligned data may be combined, such as by summing, averaging, alpha blending, maximum selection, minimum selection or other process. The combined data set is rendered as a three-dimensional representation. Separate renderings may be used, such as laying a microscopic rendering over a macroscopic rendering. The combination provides feedback about relative position of the microscopic data to the larger macroscopically scanned region.

The processor 26 may calculate quantities. Modeling and/or machine learning associated with the registered data may be performed by the processor 26.

The display 28 is a monitor, LCD, projector, plasma display, CRT, printer, or other now known or later developed devise for outputting visual information. The display 28 receives images, graphics, or other information from the processor 26, memory 12, microscopic system 14, or macroscopic system 16. The display 28 displays the images of the tissue volume.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A method for biomedical imaging, the method comprising:
obtaining microscopic data representing a first region of tissue;
obtaining macroscopic data representing a second region of tissue, the second region larger than the first region;
spatially aligning the microscopic data and the macroscopic data; and
generating an image as a function of the microscopic data, macroscopic data, or both microscopic and macroscopic data and as a function of the spatial aligning.

2. The method of claim 1 wherein obtaining microscopic data comprises obtaining confocal microscopy data representing the first region as a three-dimensional region.

3. The method of claim 1 wherein obtaining macroscopic data comprises obtaining computed tomography data, magnetic resonance data, positron emission tomography data, single photon emission tomography data, or combinations thereof.

4. The method of claim 1 wherein obtaining microscopic and macroscopic data comprises obtaining data with different imaging modalities, one of the imaging modalities having cellular, sub-cellular or molecular level resolution for the first region and another one of the imaging modalities having a less resolution for the second region, the less resolution associated with tissue structure without cellular or more detailed structure.

5. The method of claim 1 wherein obtaining microscopic data comprises obtaining in vitro or in vivo imaging data of the first region before and/or after exposure to a drug and wherein obtaining macroscopic data comprises obtaining in vivo imaging data before and/or after exposure to the drug.

6. The method of claim 1 wherein obtaining microscopic data comprises obtaining multispectral data and wherein obtaining macroscopic data comprises obtaining data responsive to imaging agent labeling of structural or functional pattern.

7. The method of claim 1 wherein obtaining microscopic and macroscopic data comprises obtaining data representing fiduciary markers.

8. The method of claim 1 wherein spatially aligning comprises registering as a function of morphological landmarks, fiduciary markers, atlases, or combinations thereof.

9. The method of claim 8 wherein registering comprises non-rigid registering.

10. The method of claim 1 wherein generating the image comprises rendering the image from the macroscopic and microscopic data, a relative position of the first region to the second region indicated in the image.

11. The method of claim 10 wherein rendering comprises rendering as a function of a zoom level, a first zoom level providing the image from macroscopic data with a sub-region representing the microscopic data, a second, greater zoom level providing the image from macroscopic and microscopic data, and a third, greatest zoom level providing the image from the microscopic data.

12. The method of claim 1 further comprising:
repeating the obtaining and spatially aligning at different times; and
determining levels of change for the macroscopic data and the macroscopic data.

13. A system for biomedical imaging, the system comprising:
a memory operable to store first data representing a tissue volume, the first data from a microscopic imaging source, and operable to store second data representing the tissue volume, the second data from a macroscopic imaging source of a different type than the microscopic imaging source, the first data having a greater resolution than the second data;
a processor operable to register the first data and the second data, and operable to render an image as a function of the first and second data; and
a display operable to display the image of the tissue volume.

14. The system of claim 13 wherein the processor is operable to render the image as a volume rendering of or an arbitrary plane through the tissue volume, the image including values for pixel locations, the values each being a function of the first and second data.

15. The system of claim 13 wherein the processor is operable to register as a function of tissue structure represented in the first and second data, fiduciary markers represented in the first and second data, functional pattern represented in the first and second data, atlas information, or combinations thereof.

16. The system of claim 13 further comprising:
a user input;
wherein the first data, the second data, or both the first and second data including labeled tissue function information, the processor operable to render the image as a function of user selection with the user input of a type of tissue function labeling.

17. The system of claim 13 further comprising:
a user input;
wherein the processor is operable to render the image as a function of a zoom level indicated by the user input, the image associated with a blending of the first and second data as a function of the zoom level.

18. In a computer readable storage medium having stored therein data representing instructions executable by a programmed processor for biomedical study, the storage medium comprising instructions for:
registering microscopy scan data with macroscopy scan data, the microscopy scan data representing a first tissue region that is a sub-set of a second tissue region represented, with lesser resolution, by the macroscopy scan data;
determining quantities from the registered microscopy and macroscopy scan data at different resolutions; and
modeling as a function of the quantities.

19. The computer readable storage medium of claim 18 further comprising instructions for navigating to regions of interest in the second tissue region at the different resolutions and rendering images for each of the different resolutions, different resolutions associated with different relative amounts of the microscopy scan data to the macroscopy scan data used in the corresponding images.

20. The computer readable storage medium of claim 18 further comprising instructions for selecting types of data representing tissue function for at least the microscopy scan data, and determining the quantities from the microscopy scan data of the selected type.

21. The computer readable storage medium of claim 18 further comprising instructions for repeating the registering and determining, the modeling being a function of a change in the quantities between repetitions.
